(19) United States
(12) Patent Application Publication
(10) Pub. No.: US 2022/0183690 A1
(43) Pub. Date: Jun. 16, 2022

(54) ORGAN RESECTION STUMP FIXING CLIP

(71) Applicants: National University Corporation Kumamoto University, Kumamoto (JP); Kyushu University, National University Corporation, Fukuoka (JP)

(72) Inventors: Hideo Baba, Kumamoto (JP); Yoichi Yamashita, Kumamoto (JP); Ijima Hiroyuki, Fukuoka (JP)

(73) Assignees: National University Corporation Kumamoto University, Kumamoto (JP); Kyushu University, National University Corporation, Fukuoka (JP)

(21) Appl. No.: 17/440,692

(22) PCT Filed: Mar. 17, 2020
(86) PCT No.: PCT/JP2020/011641

§ 371 Date: Sep. 17, 2021

(30) Foreign Application Priority Data
Mar. 18, 2019 (JP) 2019-050488

(51) Int. Cl. A61B 17/08; A61B 17/122; A61B 17/10

(52) U.S. Cl. CPC A61B 17/083; A61B 17/10; A61B 17/1227

(57) ABSTRACT

An object of the present invention is to provide a means for fixing a pancreatic resection stump created during an operation for resecting a portion of the pancreas. The present invention provides a polymeric surgical clip characterized in that the clip comprises a first arm portion, a second arm portion, and a resilient hinge portion joining the first and second arm portions together, both the arm portions are joined to the resilient hinge portion at their bases, the first arm portion has a convex outer surface and a concave inner surface clamping the pancreas, the second arm portion has a convex or flat outer surface and a concave or flat inner surface clamping the pancreas, the first arm portion forms at its distal end a deflectable hook portion curved toward the second arm portion, and the clip is configured so that, in a closed position, a distal end of the second arm portion enters inside the curved hook portion and engages with the hook portion to lock the clip in the closed position.

### TECHNICAL FIELD

The present invention relates to a clip for fixing an organ resection stump, and a method for fixing an organ resection stump using the clip.

### BACKGROUND ART

A pancreatic fistula occurring after an operation for resecting a pancreas is a phenomenon of persistent or intermittent leakage of pancreatic juice. A distal pancreatectomy is performed as a radical operation for a pancreatic body and tail tumor, but the pancreatic fistula may occur in which the pancreatic juice (digestive enzymes) leaks from the pancreatic stump after pancreatic body and tail resection, which is one of unsolved complications. The pancreatic fistula can be fatal because it sometimes dissolves surrounding tissues (an artery and the like), resulting in major bleeding. The post-operative pancreatic fistula occurs in about 30% of the distal pancreatectomies, and is also a cause of occurrence of life-threatening complications.

In the distal pancreatectomy, a main pancreatic duct of the resection stump is ligated, and pancreatic parenchyma on the pancreatic head and body side of the resection stump is closed, thereby preventing leakage of the pancreatic juice. However, damage to a pancreatic resection portion occurring at that time is thought to be a cause of the pancreatic fistula.

Various techniques have been used for closure of the pancreatic parenchyma on the pancreatic head and body side of the resection stump in the distal pancreatectomy. Stapling, teres ligament patching, fibrin sealing, TachoSil (registered trademark) patching, and a pancreatojejunostomy (pancreatogastrostomy) can be named, but there are discussions over which technique is suitable. With the increase of the distal pancreatectomies using a laparoscope, closure of the resection stump by a stapler has become more common in recent years.

A stapler used clinically in pancreatic parenchyma resection at present may cause damage to pancreatic capsule/pancreatic parenchyma when a thick pancreatic body is resected, a stapling error of the main pancreatic duct may be observed. Then, the present inventors used a reinforced Tri-Staple™ to compare it with an ordinary staple, and reported that grade B or C of the pancreatic fistulae were significantly reduced (Non-patent Document 1: Yamashita et al., Anticancer Res. 37: 1865-1868, 2017). It is reported therein that the error in the case of using the ordinary stapler was 30%, whereas the error in the case of using the reinforced Tri-Stapler was 5%.

On the other hand, a clip for ligating a blood vessel to stop a blood flow during an operation is proposed. For example, a polymeric surgical clip is proposed that comprises first and second curved leg members, both the leg members being joined at their proximal ends by a resilient hinge means, the first leg member terminating at its distal end in a deflectable hook member curved toward the second leg member, the second leg member terminating at its distal end in a complementary locking portion to the hook member (Patent Document 1: JPH1-146536A). Therein, a vessel clamping inner surface of the first leg member and a vessel clamping inner surface of the second leg member are in opposition to each other, and the radius of curvature of the inner concave surface of the first leg member is substantially the same as the radius of curvature of the inner convex surface of the second leg member. Therefore, as shown in the drawing of FIG. 7E, from the proximal ends to the distal ends of both the leg members, their vessel clamping inner surfaces are configured to overlap with each other.

In addition, a polymeric surgical clip is proposed that comprises first and second curved leg members joined by a resilient hinge means, each leg member having a vessel clamping inner surface, one of the vessel clamping inner surfaces being in opposition to the other vessel clamping inner surface, and each vessel clamping inner surface having a plurality of protrusions (Patent Document 2: JPH3-178648A). Therein, the radius of curvature of the vessel clamping inner concave surface of the first curved leg member is substantially the same as the radius of curvature of the vessel clamping inner convex surface of the second curved leg member, and, as shown in the drawing of FIG. 12E, from the proximal ends (portions proximal to the hinge means) to the distal ends of both the curved leg members, their vessel clamping inner surfaces are configured to overlap with each other.

Furthermore, a surgical clip is proposed that comprises a top jaw member and a bottom jaw member joined at a hinge section for movement around the hinge section (Patent Document 3: JP2014-534014A). Therein, the top jaw member and the bottom jaw member each include an inner convex segment in a portion of the hinge section, and are configured to clamp a proximal portion of a tissue. In addition, the top jaw member has a generally convex outer surface and an S-shaped inner surface, and the bottom jaw member has a generally concave outer surface and an S-shaped inner surface that approximately matches or conforms to the profile of the inner surface of the top jaw member. Therefore, as shown in FIG. 4, from the hinge section to the distal ends of both the jaw members, their clamping inner surfaces are configured to overlap with each other.

In addition, an anti-migration surgical ligation clip for ligating a blood vessel or tissue structure is proposed (Patent Document 4: JP2018-518271A). Therein is disclosed a surgical clip for ligating a blood vessel or tissue structure, comprising a first leg member including a first inner surface and a first plurality of protrusions disposed on the first inner surface, a second leg member including a second inner surface and a second plurality of protrusions disposed on the second inner surface, and a hinge member joining the first leg member and the second leg member, wherein at least one of the first and second plurality of protrusions includes a gable structure that extends along a longitudinal direction of the first or second inner surface. Further, as shown in FIG. 3A, the inner surfaces disposed on the respective leg members are configured to overlap with each other.

In addition, as a clip for ligating a blood vessel, a polymeric Hem-o-lok (registered trademark) is marketed by Teleflex Incorporated, and therein this clip comprises first and second curved leg members curved in the same direction, both the leg members being joined at their proximal ends by a resilient hinge, the first leg member terminating at its distal end in a deflectable hook member curved toward the second leg member, whereas the second leg member terminating at its distal end in a complementary locking portion to the hook portion. Therein, a vessel clamping inner surface of the first leg member is in opposition to a vessel clamping inner surface of the second leg member, and the radius of curvature of the inner concave surface of the first leg member is substantially the same to the radius of curvature of the inner convex surface of the second leg member. Therefore, from the proximal ends to the distal ends of both the leg members, their vessel clamping inner surfaces are configured to overlap with each other (https://www.teleflex.com/usa/en/product-areas/surgical/ligation-solutions/weck-polymer-ligation/).

Besides those, a ligation clip is proposed that comprises metallic upper and lower jaws and a hinge portion connecting the jaws together (U.S. Pat. No. 6,699,258). Therein is disclosed a clip comprising a metallic upper jaw curved outward and provided with a stress relief mechanism, and a metallic lower jaw provided at a first end with a latch portion configured to engage with an end of the upper jaw when a curved portion of the upper jaw is straightened, and provided with a hinge portion at a second end opposite the first end. In this clip, as shown in FIG. 3, when a blood vessel is closed, the curved portion of the upper jaw is straightened to permanently deform, so that the distal end of the upper jaw engages with the latch portion of the lower jaw. That is, the engagement is achieved by permanently straightening the metallic curved portion.

In addition, as a clip for an appendectomy, an AESCULAP (registered trademark) DS clip is marketed by B. Braun Aesculap Japan Co., Ltd., and therein curved metallic upper and lower jaws are straightened permanently, thereby achieving parallel closure (https://www.bbraun.jp/ja/products/b/ds-titanium-ligationclips.html).

### CITATION LIST

### Patent Document

Patent Document 1: JPH1-146536A
Patent Document 2: JPH3-178648A
Patent Document 3: JP2014-534014A
Patent Document 4: JP2014-534014A
Patent Document 5: U.S. Pat. No. 6,699,258

### Non-Patent Document

Non-Patent Document 1: Yamashita et al., Anticancer Res. 37: 1865-1868, 2017

### SUMMARY OF THE INVENTION

### Technical Problem

An object of the present invention is to provide an instrument and a means for fixing an organ resection stump created during an operation for resecting a portion of an organ, for example a pancreatic resection stump.

### Solution to Problem

The present inventors made an in-depth study diligently in order to solve the above problem, found that by using a polymeric clip having a certain shape, a resection stump of a pancreas caused by a pancreatectomy can be easily fixed, and occurrence of the pancreatic fistula can be prevented or reduced, and completed the present invention. In addition, it has been found that the present invention is also applicable to other similarly delicate organs besides the pancreas.

The present invention includes the following.

[1] A polymeric surgical clip for an organ resection (preferably a pancreatectomy) comprising a first arm portion, a second arm portion, and a resilient hinge portion joining the first and second arm portions together, both the first and second arm portions being joined to the resilient hinge portion at their bases, the first arm portion having a convex outer surface and a concave inner surface clamping an organ (preferably a pancreas), the second arm portion having a convex or flat outer surface and a concave or flat inner surface clamping the organ (preferably the pancreas), the inner surface of the first arm portion and the inner surface of the second arm portion being in opposition to each other, the first arm portion forming at its distal end a deflectable hook portion curved toward the second arm portion, the polymeric surgical clip being thereby configured so that, in a closed position, a distal end of the second arm portion enters inside the curved hook portion and engages with the hook portion to lock the clip in the closed position, the polymeric surgical clip being characterized in that

the first arm portion and the second arm portion keep substantially the same shape in an opened position and the closed position, and the inner surface of the first arm portion and the inner surface of the second arm portion clamping the organ (preferably the pancreas) are configured so that a space is created between both the inner surfaces in the closed position.

[2] The polymeric surgical clip according to the above [1], characterized in that the second arm portion has the convex outer surface and the concave inner surface clamping the organ (preferably the pancreas), the inner surface of the first arm portion and the inner surface of the second arm portion are in opposition to each other to configure opposing curved surfaces.

[3] The polymeric surgical clip according to the above [1] or [2], characterized in that a maximum distance created between the inner surface of the first arm portion and the inner surface of the second arm portion in the closed position is larger than any one of a thickness between the inner surface and the outer surface of the first arm portion or a thickness between the inner surface and outer surface of the second arm portion.

[4] The polymeric surgical clip according to any one of the above [1] to [3], wherein the thickness between the inner surface and the outer surface of the first arm portion is substantially the same as the thickness between the inner surface and the outer surface of the second arm portion.

[5] The polymeric surgical clip according to any one of the above [2] to [4], wherein the radius of curvature of the concave inner surface of the first arm portion is substantially equal to the radius of curvature of the concave inner surface of the second arm portion.

[6] The polymeric surgical clip according to any one of the above [1] to [5], wherein the first arm portion and the second arm portion each have one or two boss portions extending laterally.

[7] The polymeric surgical clip according to any one of the above [1] to [6], further having a bump portion on each of the inner surface near the base of the first arm portion and the inner surface near the base of the second arm portion.

[8] The polymeric surgical clip according to any one of the above [1] to [7], wherein the inner surface of the first arm portion and/or the inner surface of the second arm portion is an embossed surface or a surface provided with a plurality of protrusions.

[9] The polymeric surgical clip according to any one of the above [1] to [8], memorizing an overall shape of the clip in the closed position.

[10] The polymeric surgical clip according to any one of the above [1] to [9], comprising a bioabsorbable polymer.

[11] The polymeric surgical clip according to any one of the above [1] to [10], characterized by being used for fixation of a resection stump of the organ (preferably the pancreas) treated by an organ resection (preferably a pancreatectomy).

[12] The polymeric surgical clip according to the above [11], configured to clamp the pancreas at a pressure of at least 4 kPa or more, preferably 5 kPa or more, more preferably 6 kPa or more when fixing the resection stump of the pancreas.

[13] The polymeric surgical clip according to any one of the above [1] to [12], characterized by being used for a surgical method selected from the group of a distal pancreatectomy, a pancreaticoduodenectomy, an enucleation of pancreatic tumor, and a middle pancreatectomy.

[14] A method for resecting a portion of an organ (preferably a pancreas), preferably a pancreatic body and tail or a pancreatic head, characterized by fixing a portion near a cut portion of the organ (preferably the pancreas) by the polymeric surgical clip according to any one of the above [1] to [12].

[15] A surgical method based on a distal pancreatectomy or a pancreaticoduodenectomy, characterized by fixing a portion near a cut portion of a pancreas by the polymeric surgical clip according to any one of the above [1] to [13].

### Advantageous Effect of the Invention

According to the present invention are provided a clip for fixing an organ resection (preferably a pancreatic resection) stump and a method for fixing an organ resection stump (preferably a pancreatic resection stump) using the clip.

### BRIEF DESCRIPTION OF DRAWINGS

FIG. 1 is a side view of an embodiment of a surgical clip of the present invention.

FIG. 2 is a perspective view of the embodiment in FIG. 1.

FIG. 3A is a side view of the clip of the embodiment in FIG. 1 in an expanded state, and FIG. 3B is a side view of the clip in a closed state.

FIG. 4 is a perspective view of the surgical clip in FIG. 1 attached to a pancreatic resection stump.

FIG. 5 is a side view of an embodiment of a clip of the present invention having boss portions.

FIG. 6 is a perspective view of the embodiment in FIG. 5.

FIG. 7A is a side view of another embodiment of a surgical clip of the present invention, and FIG. 7B is a side view of the clip in a closed position.

FIG. 8A is a side view of another embodiment of a surgical clip of the present invention, and FIG. 8B is a side view of the clip in a closed position.

FIG. 9A is an embodiment of a clip memorizing a shape with a first arm portion and a second arm portion opened slightly, FIG. 9B is an embodiment of a clip memorizing a shape with a first arm portion and a second arm portion opened widely, and FIG. 9C is an embodiment of a clip memorizing a shape with a first arm portion and a second arm portion closed.

FIG. 10A is a side view of the clip memorizing the shape with the first arm portion and the second arm portion opened slightly, the clip being set on an applier, and FIG. 10B is a side view of the clip in an opened state.

FIG. 11 is a side view of the clip memorizing the shape with the first arm portion and the second arm portion opened widely, the clip being set on an applier.

FIG. 12 is a side view of the clip memorizing the shape with the first arm portion and the second arm portion closed, the clip being set on an applier. It should be noted that FIG. 12 shows the clip being set on the applier after a distal end of the first arm portion and a distal end of the second arm portion are disengaged in order to facilitate a clip opening operation.

FIG. 13 is a side view of an example of an applier (closer) used in order to put into a closed state the clip memorizing the shape with the first arm portion and the second arm portion closed (to lock the distal end of the first arm portion and the distal end of the second arm portion) after clamping a pancreas by the clip.

### Description of Embodiments

Hereinafter, the present invention will be illustrated with reference to the exemplary embodiments along with preferred methods and materials which can be used in practice of the present invention. Unless otherwise specified in the sentences, any technical terms and scientific terms used in the present specification have the same meaning as those generally understood by those of ordinary skill in the art to which the present invention belongs. Any materials and methods equivalent or similar to those described in the present specification can be used for practicing the present invention. All publications and patents cited herein in connection with the present invention described herein are incorporated by reference, for example, as indicating methodology, materials, etc. that can be used in the present invention.

In the present specification, when the expression “X to Y” is used, it means that X is included as a lower limit and Y as an upper limit, or that X is included as an upper limit and Y as a lower limit. In the present specification, “about” is used to mean that ±10% is allowed.

Though a surgical clip of the present invention is described below in detail, taking as an example a case where the clip is applied to a pancreas, the surgical clip of the present invention is usable for other similarly delicate and thick organs beside the pancreas, for example, a liver, a lung, an appendix, and an ovary. Therefore, the clip of the present invention is a clip for fixing a resection stump of a cut portion of an organ selected from a pancreas, a liver, a lung, an appendix, or an ovary, preferably a clip for fixing a resection stump of a pancreas.

The surgical clip of the present invention is provided with a first arm portion, a second arm portion, and a resilient hinge portion joining the first and second arm portions together, and both the first and second arm portions are joined together via the hinge portion at their bases. The first and second arm portions are configured to engage with each other at their terminating ends (distal ends) when the clip is in a closed position. The hinge portion is resilient, and allows the first arm portion and the second arm portion to move to an opened position, and to move from the opened position to the closed position. Each arm portion has an inner surface opposing an inner surface of the other arm portion. The inner surface of the first arm portion is concave. The inner surface of the second arm portion is concave or flat, preferably concave. This prevents their inner surfaces from closely overlap with each other even in the closed position where the terminating ends of the first arm portion and the second arm portion are engaged with each other, so that a space is secured between the inner surfaces of the first arm portion and the second arm portion. Therefore, even when a pancreas, which is a delicate organ, is clamped, excessive damage to the organ can be prevented.

The first arm portion has a hook portion at its terminating end, and the hook portion is deflectable (having a resiliently bending property) and curved toward the second arm portion. Since the hook portion is flexible, when the first and second arm portions move around the hinge portion from the opened position to the closed position, the distal end of the second arm portion abuts against the above hook portion, and when both the arm portions move further to the closed position, the hook portion deflects toward the outside of the first arm portion, so that the distal end of the second arm portion enters inside the hook portion of the first arm portion. This causes an outer surface of the distal end of the second arm portion to engage with the inside of the hook portion of the first arm portion, thereby locking the clip in the closed position.

The curved shape of the hook portion includes both gently-curving and sharply-curving shapes, and further includes a form that can be called a bend, but preferably it is a sharply curved state or a bent state. This forms a good state of engagement (state of locking) between the first arm portion and the second arm portion, and thus the clip can be kept in a good closed state. It should be noted that, if it is desired that an excessive load on the hook portion be avoided, or if it is desired that a space between the first and second arm portions be increased, or in other cases, a gentle curve can be used.

It is possible to curve the inner and outer surfaces of the hook portion continuously, and this prevents occurrence of excessive stress concentration in a corner portion.

The clip of the present invention can be provided with one or two cylindrical boss portions extending laterally, in a distal position from the hinge portion in, each of the first arm portion and the second arm portion, preferably two on both side surfaces. Since the boss portions are provided, when the clip is set on an applier, the clip can be stably held on the applier, and simultaneously an opening and closing operation of the clip can be facilitated by using the applier.

The surgical clip of the present invention can be used for fixation of a resection stump of a pancreas. Though not limited thereto, the clip of the present invention can be used in a pancreatectomy, for example, a distal pancreatectomy, a pancreaticoduodenectomy, a middle pancreatectomy, a tumor enucleation, preferably for a distal pancreatectomy. The surgical clip of the present invention can be used in order to fix pancreatic parenchyma, but preferably it is used in order to fix pancreatic parenchyma on a pancreatic head and body side after resection. It is preferred that a main pancreatic duct of a resection stump be ligated prior to fixing by the clip, but ligation does not have to be performed.

In addition, the surgical clip of the present invention can be used for fixation of a resection stump of a pancreas in a laparoscopic pancreatectomy by using it together with an applier.

Preferred embodiments of the surgical clip of the present invention are described below with reference to the drawings. It should be noted that the present invention is not limited to the embodiments described below.

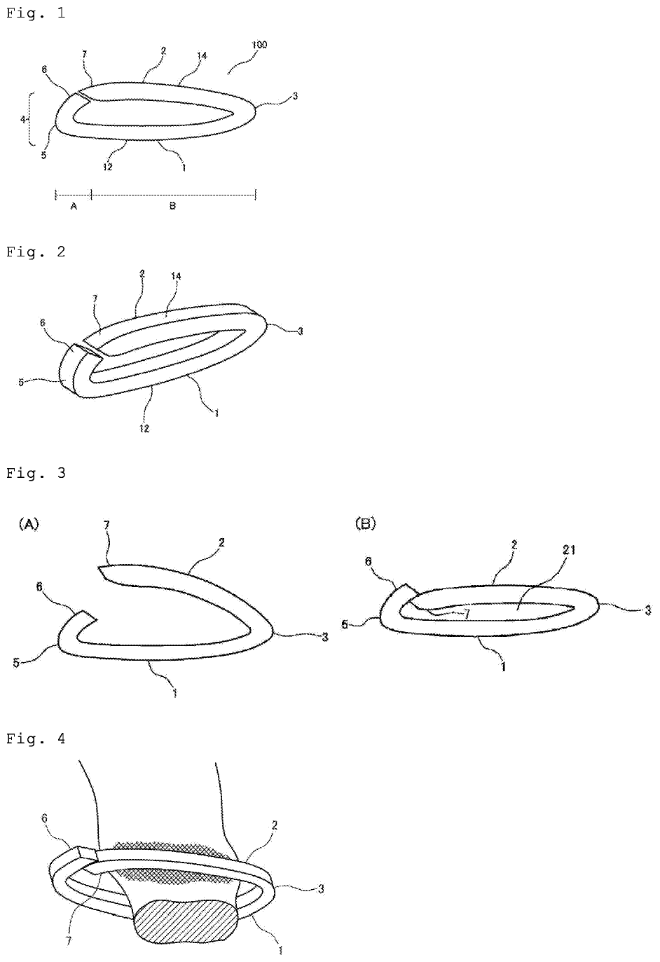

FIG. 1 shows an embodiment of the surgical clip of the present invention. The clip of the present invention that can be used for fixation of a resection stump of a pancreas is composed of a first arm portion 1 and a second arm portion 2, and a hinge portion 3 connecting both the arm portions together at their bases. The hinge portion 3 is resilient. Therefore, the first arm portion and the second arm portion can be opened from a position shown in FIG. 1 to a position shown in FIG. 3A, and can be moved to a closed position shown in FIG. 3B. This enables fixation of a pancreas by holding the pancreas between the first arm portion and the second arm portion in an opened state shown in FIG. 3A, and then moving the first arm portion and the second arm portion to a closed state shown in FIG. 3B. Consequently, when a pancreas is cut, a pancreatic portion left near a cut portion of the pancreas (in the case of resection of a pancreatic body and tail, a pancreatic head side of the cut portion) can be fixed. Resection of a pancreas can be performed by a method in which after a portion to be left near a cut portion of the pancreas is fixed by the clip of the present invention, cutting is performed to resect a removal portion, or by a method in which after a pancreas is cut to resect a removal portion, a portion near the cut portion is fixed, but preferably cutting is performed after fixation by the clip. FIG. 4 is a drawing showing the clip fixing a resection stump of a pancreas after resection.

The first arm portion 1 has a concave inner surface 11 and a corresponding convex outer surface 12, and a hook portion 4 is disposed on its terminating end (distal end) side. The second arm portion 2 has a concave or flat inner surface 13 and a convex or flat outer surface 14, and terminates in a distal end 7 engaging with the hook portion 4.

The hook portion 4 is curved toward the second arm portion, and comprises a curved portion 5 and a distal end 6. The hook portion 4 is deflectable, and configured so that, when the first and second arm portions move around the hinge portion from the opened position to the closed position, the above hook portion 4 deflects toward the outside of the first leg member, and the distal end 7 of the second arm portion enters inside the above curved hook portion, and engages with the hook portion 4 to lock the clip in the closed position. In the closed state, the distal end 6 and the distal end 7 are put into an engaged state, thereby fixing the first arm portion and the second arm portion in the closed position.

In any state of the clip of the present invention, for example, in the opened state or the closed state, the first arm portion and the second arm portion are unchanged in shape, the same or substantially the same in shape. Therefore, in the closed state, the inner surface of the first arm portion is kept in a concave shape, whereas the inner surface of the second arm portion is kept in a concave or flat (preferably concave) shape, and thus a space can be secured between both the inner surfaces.

The inner shape of the hook portion 4 and the shape of the distal end 7 of the second arm portion are not particularly limited as long as they can engage with each other to fix a resection stump of a pancreas in the closed state. The shape of the curved portion 5 of the hook portion 4 includes both forms of a gentle curve and a sharp curve, and even a form that can be called a bend, but preferably it is a sharply-curved or bent state. It should be noted that, if it is desired that an excessive load on the hook portion be avoided, or if it is desired that a space between the first and second arm portions be increased, or in other cases, a gentle curve may be used. On the other hand, it is preferred that the shape of the distal end 7 of the second arm portion 2 be such a shape that the distal end 7 will easily enter inside the curved portion 5 in a curved or bent state and will not disengage while a pancreas is fixed, and, for example, it includes a shape decreasing in thickness toward its distal end. The hook portion 4 and the distal end 7 each may be provided with, for example, a claw in order to prevent them from disengaging after engagement. The hook portion 4 and the distal end 7 take these shapes, thereby facilitating fixation of a pancreatic head side of a cut portion of a pancreas, and simultaneously forming good engagement (state of locking) therebetween, so that the clip can keep a good closed state with the pancreas clamped therein.

In a preferred embodiment, a thickness between the inner surface 11 and the outer surface 12 in an almost entire portion of the first arm portion (namely most portions excluding the distal end and the base) is, as a whole, substantially constant throughout its length. In addition, in a preferred embodiment, a thickness between the inner surface 13 and the outer surface 14 in an almost entire portion of the second arm portion (namely most portions excluding the distal end and the base) is, as a whole, substantially constant throughout its length. In addition, it is preferred that the thickness of the first arm portion and the thickness of the second arm portion be substantially the same thickness. The thickness of the first arm portion or the second arm portion is not particularly limited, and can be selected appropriately according to the material used, but it is preferably 2 to 10 mm, more preferably 2 to 7 mm, even more preferably 2 to 6 mm.

The inner surface side 11 of the first arm portion is concave, and the radius of curvature of the concave inner surface can be selected appropriately according to the size and/or shape of an object to be fixed. The inner surface side 13 of the second arm portion is concave or flat, but preferably concave. When the inner surface side of the second arm portion is concave, the radius of curvature of the concave inner surface can be selected appropriately according to the size and/or shape of an object to be fixed. Since the inner surface 11 is concave, whereas the inner surface 13 is concave or flat, when the hook is closed, as shown in FIG. 3B, the inner surfaces do not overlap with each other in parallel, so that a space 21 is secured between the inner surfaces. This enables prevention of excessive damage to an organ even which is a delicate organ, such as a pancreas is clamped. In a case where the inner surface sides of the first and second arm portions are both concave, it is preferred that their radius of curvature be the same or substantially the same. The distance of the space 21 between the inner surfaces in the closed position is selected appropriately according to the size and/or shape of an object to be fixed. Actually, the thickness of a pancreatic cut portion is 0.5 to 4 cm, usually 1 to 2 cm, and therefore, according to the thickness of a target cut portion, a clip is selected appropriately that has such a degree of distance of the space 21 that the clip can close the entire cut portion securely to some degree, but not too strongly. Though not limited thereto, a maximum width between the inner surfaces in the space 21 has a lower limit of preferably about 1 mm, more preferably about 2 mm, even more preferably about 3 mm, and has an upper limit of preferably about 15 mm, more preferably about 10 mm, even more preferably about 8 mm, still even more preferably about 5 mm. The thickness of a pancreas is usually 1 to 2 cm, and therefore the clip having the space 21 having a maximum width of about 3 to 5 mm is most versatile.

The length of the clip of the present invention is not particularly limited, and can be selected appropriately according to the size and/or shape of an object to be fixed. Though not limited thereto, it is preferably 3 to 15 cm, more preferably 4 to 12 cm, even more preferably 5 to 10 cm.

The size of the hook portion 4 in the first arm portion of the clip of the present invention is not particularly limited, but the ratio of the hook portion 4 to the other portion (ratio of A:B) is preferably 1:2 to 1:10, more preferably 1:3 to 1:8, even more preferably 1:5to 1:7.

The clip of the present invention can be provided with an anti-slip mechanism on the inner surface of the first arm portion and/or the inner surface of the second arm portion in order to prevent the clip from slipping out of place and/or slipping off after the clip is fixed to a pancreas. The anti-slip mechanism is not particularly limited as long as it can prevent the clip from slipping out of place, and any mechanism may be used, and, for example, the anti-slip mechanism can be achieved by embossing the inner surface, or by providing the inner surface with a plurality of protrusions.

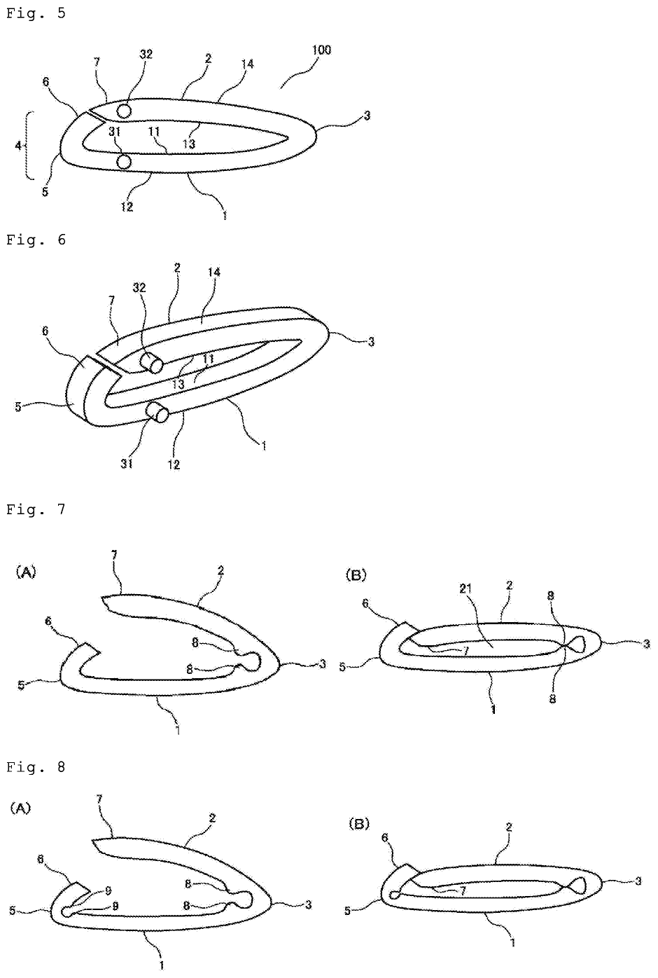

Another embodiment of the clip of the present invention is shown in FIGS. 5 and 6. The clip of the present invention can be provided with laterally-extending cylindrical boss portions 31, 32 on the first arm portion and the second arm portion, respectively, in a distal position from the hinge portion 3. The arm portions each can be provided with one or two boss portions, but preferably two boss portions. In a case where the arm portions are each provided with two boss portions, it is preferred that one boss portion be provided on each side of the arm portion so as to form a pair. In a case where the arm portions are each provided with one boss portion, it is preferred that the boss portions be provided in a pair on the same sides of the arm portions. The length and/or size of the boss portion is not particularly limited as long as the boss portion can engage with a catching mechanism of an applier.

The clip of the present invention can also be manufactured using a shape-memory plastic material. The shape memorized in such a case is not particularly limited. It can be, for example, a clip memorizing a shape with the first arm portion and the second arm portion opened slightly, such as shown in FIG. 9A, a clip memorizing a shape with the first arm portion and the second arm portion opened widely, such as shown in FIG. 9B, or a clip memorizing a shape with the first arm portion and the second arm portion closed, such as in FIG. 9C. Using such a clip memorizing a shape in an opened state as shown in FIG. 9B enables holding a pancreas easily, whereas using such a clip memorizing a shape in a closed state as shown in FIG. 9C enables keeping the closed state more stably.

Such a clip having a shape memory property in a closed position as shown in FIG. 9C at from room temperature to body temperature, preferably at body temperature, is preferred because it makes the fixing operation easier, and can keep the closed state more stably for a long period of time after fixation. Though not limited thereto, the clip having a shape memory property in a closed position at body temperature can be used, for example in the following manner. First, the clip is cooled at room temperature or lower, preferably cooled with ice, in an expanded state (opened state), to prevent closure and fixation due to body temperature (prevent a change into the memorized shape). In this state, the clip is attached to a pancreas, and the clip is closed, thereby causing transition into the closed state. More preferably, the clip in an opened state is stored in advance in a container shaped like the clip in the opened state, and the clip is cooled until use or before use. Cooling can be performed, for example, by placing the clip in a refrigerator or on ice together with the container keeping the clip therein. At the point of use, the clip is taken out of the container and used. When the clip is attached to a pancreas, the clip is warmed to body temperature, and thus the clip is fixed in shape to the closed position that is the memorized shape. In order to promote shape fixation, a saline solution warmed to body temperature may also be poured over the clip.

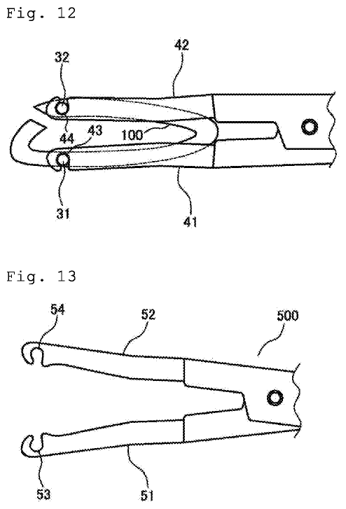

The clip of the present invention can also be used with an applier, which is particularly useful in the case of a laparoscopic operation. Though an example of using an applier is described with reference to FIGS. 10 to 13, the shape and/or use form of an applier is not limited thereto.

FIGS. 10A and 10B are an example of using a clip with the first arm portion and the second arm portion opened slightly, such as shown in FIG. 9A. FIG. 10A shows a clip 100 attached to an applier 400 at the point of use. The applier 400 can set thereon the clip 100 in a holding state in a position between a leg portion 41 and a leg portion 42. It is preferred that the applier take a scissors shape so that it can open the clip. Inside the leg portions 41 and 42 are provided catching mechanisms 43 and 44, respectively. The catching mechanisms 43 and 44 can engage with boss portions 31 and 32 of the clip, respectively, to fix the clip. The catching mechanism provided on each leg portion of the applier is one or two in number, but preferably two in number. It is preferred that the catching mechanism take a U-shaped structure opened toward a distal end of the leg portion. This can prevent the clip from dropping off when the clip approaches a pancreas within an abdominal cavity with the clip fixed to the applier.

FIG. 10B is an enlarged view showing the boss portion 32 of the clip and the catching mechanism 44 of the applier in their engaged state when the clip is set on the applier.

After the clip is set on the applier, the applier of the scissors shape is opened, and thereby the clip can be operated into a further opened state. The clip, in the further opened state, tries to return to its original shape, so that opposite forces act on the catching mechanism of the applier and the boss portion of the clip, and thus the clip can be prevented from dropping off from the applier when the clip opened approaches a pancreas. The clip opened is set on a pancreatic head side of a cut site of the pancreas, and thereafter the applier is closed, thereby locking the clip in the closed state.

FIG. 11 is an example of using a clip with the first arm portion and the second arm portion opened widely, namely in an opened state, such as shown in FIG. 9B. FIG. 11 shows a clip 100 attached to an applier 400 at the point of use. The applier 400 can set thereon the clip 100 in a holding state in a position between a leg portion 41 and a leg portion 42. It is preferred that catching mechanisms 43, 44 of the applier take a U-shaped structure opened inward. When the applier is closed, the clip tries to return to its original shape, so that opposite forces act on the catching mechanism of the applier and the boss portion of the clip, and thus the clip can be prevented from dropping off when the clip approaches a pancreas within an abdominal cavity. The clip opened is set on a pancreatic head side of a cut site of the pancreas, and thereafter the applier is closed, thereby locking the clip in the closed state.

FIG. 12 is an example of using a clip with the first arm portion and the second arm portion closed, such as shown in FIG. 9C. FIG. 12 shows the clip set on an applier after the distal end of the first arm portion and the distal end of the second arm portion are disengaged in order to facilitate the clip opening operation. An applier 400 can set thereon a clip 100 in a holding state in a position between a leg portion 41 and a leg portion 42. It is preferred that the catching mechanisms 43, 44 of the applier take a U-shaped structure opened outward. When the applier is opened, the clip tries to return to its original shape, so that opposite forces act on the catching mechanism of the applier and the boss portion of the clip, and thus the clip can be prevented from dropping off when the clip approaches a pancreas within an abdominal cavity. The clip opened is set on a pancreatic head side of a cut site of the pancreas, and thereafter the applier is closed. Locking the clip in the closed state can be performed using an applier, such as shown in FIG. 12, but, if locking is insufficient, releasing the applier from the clip, and then the clip may also be locked by using a closer, such as shown in FIG. 13. The closer is provided with receiving portions 53, 54 abutting against the boss portions of the clip.

Another embodiment is described below in which a clip is used that memorizes a shape with the first arm portion and the second arm portion closed, such as shown in FIG. 9C. An example is shown in which a clip is used that memorizes a shape in a closed position, such as shown in FIG. 9C, for example at about 30 to 38° C., preferably at about 37° C. that is body temperature. Since the clip in the closed state cannot be made to approach a pancreas within an abdominal cavity, the clip needs to be put into a widely opened state. First, the clip, in a widely expanded state, is cooled to room temperature or lower, preferably cooled to about 4° C. or lower. Cooling to about 4° C. or lower can be performed, for example by cooling the clip in an expanded state with ice. Alternatively, the clip in an opened state is put into a container shaped like the clip in the opened state, and the clip is refrigerated or cooled together with the container until use or before use. This can prevent the clip from being warmed to body temperature to change into the closed state (change into the memorized shape), and thus the clip can be kept in a widely opened state. A condition for prevention of such a change into the memorized shape can be selected appropriately according to the clip material and the like. Next, the clip opened widely is attached to an applier, approaches a pancreas, and is set on a pancreatic head side of a cut site of the pancreas. If necessary, in order to prevent the temperature of the clip from rising, the applier may be cooled in advance, or the applier may be provided with a cooling mechanism. The clip set on the pancreas is warmed to body temperature, and thus tries to return to the memorized shape. This facilitates the operation of using the applier to lock the clip in the closed state. In addition, in order to promote the clip returning to its original shape, if necessary, a saline solution warmed to body temperature may also be poured over the clip.

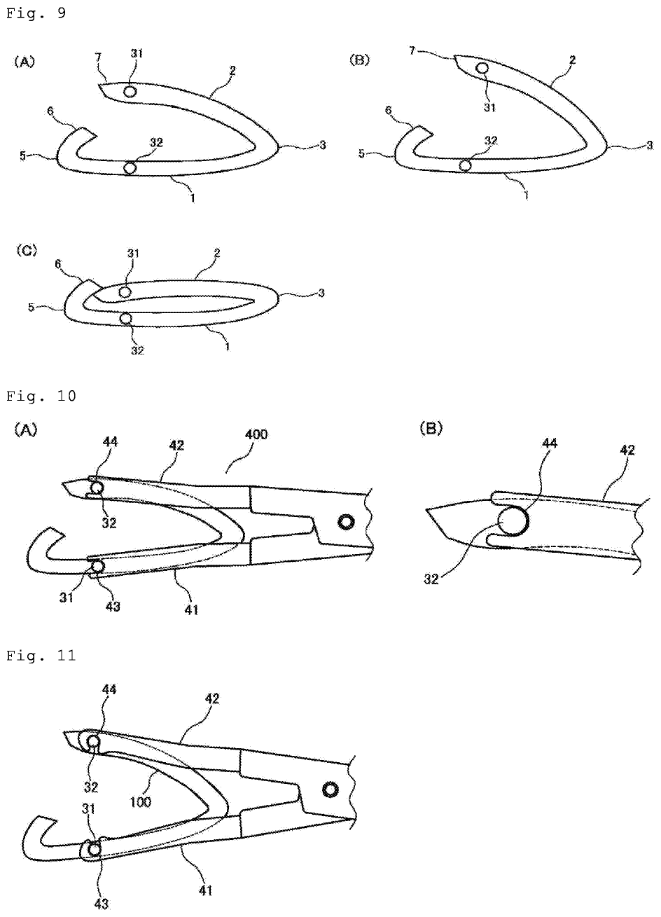

Another embodiment of the clip of the present invention is shown in FIGS. 7A and 7B. Therein, the hinge portion 3 is of a larger radius of curvature, and simultaneously the clip has an inward bump-like structure 8 near the base of each arm portion. The hinge portion 3 takes a larger radius of curvature, thereby enlarging an opening portion between the first arm portion and the second arm portion, thus producing the effect of facilitating clamping of a pancreas more or enabling clamping of a thicker pancreas. In addition, as shown in FIG. 7B, the two bump-like structures 8 come into contact with each other in the closed state, thereby enabling the inner surfaces of the arm portions of the clip to apply a uniform force to the pancreas throughout from the hinge portion to the distal ends, resulting in sufficient fixation of the pancreas.

Another embodiment of the clip of the present invention is shown in FIG. 8. Therein, the hinge portion 3 is of a larger radius of curvature, simultaneously the clip has an inward bump-like structure 8 near the base of each arm portion, furthermore the curved portion 5 of the first arm portion is a gentler curve, and simultaneously two bump-like structures 9 are on the distal end of the first arm portion. Since the curved portion 5 is a gentler curve, not only molding is facilitated, but also the clip opening and closing operation is facilitated more. In addition, the opening portion between the first arm portion and the second arm portion becomes large, the space 21 between the inner surfaces can be designed to be larger, and thus this clip is suitable for fixing a thick pancreatic body.

When a resection stump of a pancreas is fixed by the clip of the present invention, a main pancreatic duct, and further an accessary pancreatic duct, if necessary, of a pancreatic closure portion are closed, and thus leakage of pancreatic juice from the pancreatic stump (a pancreatic fistula) can be prevented. In order to prevent leakage of pancreatic juice, the main pancreatic duct needs to be closed by the clip so that the pressure-resistant performance of the main pancreatic duct is equal to or more than a certain level. It is desired that the pressure-resistant performance of the main pancreatic duct required in order to prevent leakage of pancreatic juice to prevent the pancreatic fistula be at least about 4 kPa or more, preferably about 5 kPa or more, more preferably about 6 kPa or more. Therefore, the pancreatic fistula can be prevented by fixing the resection stump of the pancreas by the clip of the present invention to such an extent that a desired pressure-resistant performance of the main pancreatic duct can be achieved. Therefore, it is desired that the clip of the present invention be configured to clamp a pancreas at a pressure of at least about 4 kPa or more, preferably about 5 kPa or more, even more preferably about 6 kPa or more, when fixing a resection stump of the pancreas, whereas it is desired that the clip be configured to clamp a pancreas at such a degree of pressure, as the upper limit, as not to cause necrosis of a closure portion of the pancreas clamped by the clip of the present invention.

It is desired that the clip of the present invention be manufactured as a plastic integrally-molded article. The plastic material is not particularly limited as long as it has biocompatibility, but preferably it is a commercially available biocompatible plastic or biodegradable plastic of a relatively high strength. Examples of preferred plastics are those used for a surgical implant operation. The plastic material can be selected freely from these plastic materials according to the purpose. Though not limited thereto, biocompatible materials, for example, can include polyethylene, Teflon (registered trademark), polyoxymethylene, polyurethane, polydioxanone, poly(ε-caprolactone), polyvinyl chloride, polypropylene, polymethylmethacrylate, polystyrene, and their derivatives, and biodegradable or bioabsorbable materials, for example, can include polyglycolic acid, polylactic acid, and their copolymers, polydioxanone, and poly(ε-caprolactone), as well as their derivatives. In addition, the plastic materials can include a blend of a plurality of any of the above materials in any ratio.

In a case where the clip of the present invention is given a shape memory property, the above plastic materials or their derivatives having a shape memory property may be used, or a plurality of any of the materials blended in any ratio may also be used.

The surgical clip of the present invention can be manufactured using a publicly-known resin molding means, for example, it can be manufactured using injection molding or extrusion molding.

Though the present invention has been described as above, taking resection of a pancreas by way of example, the surgical clip of the present invention is also applicable, in addition to the pancreas, to a liver, a lung, an appendix, and/or an ovary, which are delicate and thick organs like the pancreas. As for the clip of the present invention used in resection of a liver, a lung, an appendix, and/or an ovary, the distance between the inner surfaces of both the arm portions, the length and thickness of the arm portion, the ratio of the hook portion to the arm portion, and the other configurations may be selected appropriately according to the kind of a target organ, the size and state of a portion to be resected, and the other conditions.

### EXAMPLE

The surgical plastic of the present invention was produced in the following manner.

A mold of the clip was made of silicone rubber. Polycaprolactone whose molecular weight was about 80,000 and which had an excellent elastic modulus was heated to melt, and loaded into the mold. By natural heat dissipation and cooling, a clip having an intended shape was made.

Using the clip made above, a pancreatectomy was performed on a pig. The abdomen of the pig (a distal pancreatectomy model, two subjects) was opened, and, under visual control, the clip was attached to the body of the pancreas. Thereafter, the tail side was resected by Metzenbaum scissors. A month after the operation, the pig was sacrificed and checked for a pancreatic fistula, and no pancreatic fistula was observed in the abdominal cavity. In addition, a pancreatic portion fixed by the clip of the present invention had been kept in a good state.

### INDUSTRIAL APPLICABILITY

The surgical clip of the present invention can be used to fix a resection stump of a pancreas.

### DESCRIPTION OF REFERENCE SIGNS

1 . . . first arm portion

2 . . . second arm portion

3 . . . hinge portion

4 . . . hook portion

5 . . . curved portion

6 . . . distal end of first arm portion

7 . . . distal end of second arm portion

8, 9 . . . bump-like structure

11 . . . inner surface of first arm portion

12 . . . outer surface of first arm portion

13 . . . inner surface of second arm portion

14 . . . outer surface of second arm portion

21 . . . space between inner surfaces

31, 32 . . . boss portion

41, 42 . . . leg portion of applier

43, 44 . . . catching mechanism

51, 52 . . . leg portion of closer

53, 54 . . . receiving portion

100 . . . clip

400 . . . applier

500 . . . closer

1. A polymeric surgical clip for a pancreatectomy comprising a first arm portion, a second arm portion, and a resilient hinge portion joining the first and second arm portions together, both the first and second arm portions being joined to the resilient hinge portion at their bases, the first arm portion having a convex outer surface and a concave inner surface clamping a pancreas, the second arm portion having a convex or flat outer surface and a concave or flat inner surface clamping the pancreas, the inner surface of the first arm portion and the inner surface of the second arm portion being in opposition to each other, the first arm portion forming at its distal end a deflectable hook portion curved toward the second arm portion, the polymeric surgical clip being thereby configured so that, in a closed position, a distal end of the second arm portion enters inside the curved hook portion and engages with the hook portion to lock the clip in the closed position, the polymeric surgical clip being characterized in that the first arm portion and the second arm portion keep substantially the same shape in an opened position and the closed position, and the inner surface of the first arm portion and the inner surface of the second arm portion clamping the pancreas are configured so that a space is created between both the inner surfaces.

2. The polymeric surgical clip according to claim 1, characterized in that the second arm portion has the convex outer surface and the concave inner surface clamping the pancreas, the inner surface of the first arm portion and the inner surface of the second arm portion are in opposition to each other to configure opposing curved surfaces.

3. The polymeric surgical clip according to claim 1, characterized in that a maximum distance created between the inner surface of the first arm portion and the inner surface of the second arm portion in the closed position is larger than any one of a thickness between the inner surface and the outer surface of the first arm portion or a thickness between the inner surface and outer surface of the second arm portion.

4. The polymeric surgical clip according to claim 1, wherein the thickness between the inner surface and the outer surface of the first arm portion is substantially the same as the thickness between the inner surface and the outer surface of the second arm portion.

5. The polymeric surgical clip according to claim 1, wherein the radius of curvature of the concave inner surface of the first arm portion is substantially equal to the radius of curvature of the concave inner surface of the second arm portion.

6. The polymeric surgical clip according to claim 1, wherein the first arm portion and the second arm portion each have one or two boss portions extending laterally.

7. The polymeric surgical clip according to claim 1, further having a bump portion on each of the inner surface near the base of the first arm portion and the inner surface near the base of the second arm portion.

8. The polymeric surgical clip according to claim 1, wherein the inner surface of the first arm portion and/or the inner surface of the second arm portion is an embossed surface or a surface provided with a plurality of protrusions.

9. The polymeric surgical clip according to claim 1, memorizing an overall shape of the clip in the closed position.

10. The polymeric surgical clip according to claim 1, comprising a bioabsorbable polymer.

11. The polymeric surgical clip according to claim 1, characterized by being used for fixation of a resection stump of the pancreas treated by a pancreatectomy.

12. The polymeric surgical clip according to claim 11, configured to clamp the pancreas at a pressure of at least 4 kPa or more when fixing the resection stump of the pancreas.

13. The polymeric surgical clip according to claim 1, characterized by being used for a surgical method selected from the group of a distal pancreatectomy, a pancreaticoduodenectomy, an enucleation of pancreatic tumor, and a middle pancreatectomy.
